SEQUENCE LISTING (8 sequences)

SEQ ID NO: 1 - DNA, 20 bp, Artificial
  Synthetic oligonucleotide
ctgcacacagaagtccctga

SEQ ID NO: 2 - DNA, 20 bp, Artificial
  Synthetic oligonucleotide
ccttgtcgccactagaaagc

SEQ ID NO: 3 - DNA, 20 bp, Artificial
  Synthetic oligonucleotide
gcagtccaaaattgagaaga

SEQ ID NO: 4 - DNA, 20 bp, Artificial
  Synthetic oligonucleotide
ctgttgctgcccatttgaag

SEQ ID NO: 5 - DNA, 19 bp, Artificial
  Synthetic oligonucleotide
ttcagcccttgcttgcctc

SEQ ID NO: 6 - DNA, 22 bp, Artificial
  Synthetic oligonucleotide
acacttttactccgaagtcggt

SEQ ID NO: 7 - DNA, 21 bp, Artificial
  Synthetic oligonucleotide
aggtcggtgtgaacggatttg

SEQ ID NO: 8 - DNA, 23 bp, Artificial
  Synthetic oligonucleotide
tgtagaccatgtagttgaggtca

(19) United States
(12) Patent Application Publication
(10) Pub. No.: US 2006/0234911 A1
(43) Pub. Date: Oct. 19, 2006

(54) Method of reversing epithelial mesenchymal transition

(76) Inventors: F. Michael Hoffmann, Madison, WI (US); Shreyasi Das, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/388,433

(22) Filed: Mar. 24, 2006

Related U.S. Application Data
(60) Provisional application No. 60/664,993, filed on Mar. 24, 2005.

(51) Int. Cl. A61K 38/16

(52) U.S. Cl. 514/2

(57) ABSTRACT

A method of reversing epithelial mesenchymal transition, comprising the step of treating a fibrotic disease patient or cancer disease patient with an amount of kinase inhibitor capable of reversing EMT, wherein the kinase inhibitor comprises a TGF-βI kinase inhibitor and a Rho kinase inhibitor or a TGF-βI inhibitor and a p38 MAPK inhibitor is disclosed.

### CROSS REFERENCE TO RELATED APPLICATIONS

This application claims the benefit of U.S. Ser. No. 60/664,993, filed Mar. 24, 2005, incorporated by reference herein.

### STATEMENT OF FEDERALLY SPONSORED RESEARCH

This invention was made with United States government support awarded by the following agencies: NIH CA090875. The United States has certain rights in this invention.

### BACKGROUND OF THE INVENTION

Epithelial to mesenchymal transition (EMT) is defined by loss of epithelial cell morphology, dissociation of cell-cell contacts, reduction in proteins mediating cell-cell contacts, remodeling of the actin cytoskeleton, and acquisition of mesenchymal cell shape (Savagner P (2001) Bioessays 23:912-923; Thiery J P (2003) Curr Opin Cell Biol 15:740-746.) During EMT, cells diminish epithelial gene expression and acquire mesenchymal gene expression (Strutz, et al. (2002) Kidney Int 61(5): 1714-28.) Cortical actins, the dense actin filament bundles that lie under the plasma membrane, reorganize or are lost, while stress fibers comprising F-actin are gained. In normal development, EMT has been associated with processes in gastrulation, heart formation, somitogenesis, palate formation and Mullerian tract regression (Savagner P (2001) (supra); Shook D and Keller R (2003) Mech Dev 120:1351-1383). EMT also has been causally linked to tumor invasion and metastasis (Gotzmann J, et al. (2004) Mutat Res 566:9-20). In renal fibrosis, tubular epithelial cells undergo EMT in response to primary insults such as hypertension or diabetes, resulting in the production and deposition of excessive extracellular matrix proteins (Zeisberg M and Kalluri R (2004a) Blood Purif 22:440-445).

The suppression of TGFβ1 either by neutralizing antibodies or chemical inhibitors ameliorates the progression of EMT, thereby blocking the damaging results of TGFβ1 in organ fibrosis (Border, W. A. and N. A. Noble (1997) Kidney Int 51(5): 1388-96;.Bottinger E P and Bitzer M (2002) J Am Soc Nephrol 13:2600-2610; Dai C, et al. (2003) J Biol Chem 278:12537-12545; Schnaper H W, et al. (2003) Am J Physiol Renal Physiol 284:F243-252.) The role of TGF-β as a key mediator of fibrosis has stimulated the development of a number of therapeutic strategies, some of which are in clinical trials, including soluble type II TGF-β receptor (SRF2), neutralizing antibodies to TGF-β (Lerdelimumab, Metelimumab and GC-1008), antisense to TGF-β (AP-12009 and AP-11014), and small molecule inhibitors of the TGF-β Type I receptor kinase (Yingling J M, et al. (2004) Nat Rev Drug Discov 3:1011-1022).

Several inhibitors targeting the ATP-binding site of the TGF-β type I receptor kinase have been described recently including SB431542 (Callahan, J. F., et al. (2002) J Med Chem 45(5): 999-1001; Inman, G. J., et al. (2002) Mol Pharmacol 62(1): 65-74; Laping, N. J., et al. (2002) Mol Pharmacol 62(1): 58-64), NPC-30345 (Kim, D. K., J. Kim, et al. (2004) Bioorg Med Chem 12(9): 2013-20), BIBU 3039 (Eger A, et al. (2004) Oncogene 23:2672-2680), and series of substituted pyrazole and dihydropyrrolopyrazole compounds (Sawyer, J. S., et al. (2004) Bioorg Med Chem Lett 14(13): 3581-4; Yingling J M, et al. (2004) Nat Rev Drug Discov 3:1011-1022). For example, SB431542, a pyridinyl-imidazol compound, is a potent inhibitor of TβRI kinase activity that, as a consequence, blocks the phosphorylation of Smad2 and Smad3 (Inman, G. J., et al. (2002) Mol Pharmacol 62(1): 65-74) and inhibits TGF-β-induced cell proliferation and motility of glioma cells (Hjelmeland, M. D., et al. (2004) Mol Cancer Ther 3(6): 737-45) and fibrosis in skin fibroblasts (Mori, Y., et al. (2004) Arthritis Rheum 50(12): 4008-21.) In addition to inhibitors of the TGF-β type I receptor kinase, small molecule inhibitors of several other kinases also block EMT or cell phenotypes related to EMT. Inhibitors of p38 mitogen activated protein kinase (p38 MAPK), phosphatidylinositol 3-kinase (PI3K), MAP kinase and RhoA kinase, implicate these cellular signaling factors, in the transition to the mesenchymal state (Bakin A V, et al. (2002) J Cell Sci 115:3193-3206; Bakin A V, et al. (2000) J Biol Chem 275:36803-36810; Bhowmick N, et al. (2001 a) J Biol Chem 5:5; Janda E, et al. (2002) J Cell Biol 156:299-313; Xie L, et al. (2004) Neoplasia 6:603-610.) For example, the chemical inhibitor of p38 MAPK, SB203580, binds to the ATP-binding site of p38a and p38β (Fitzgerald, C. E., et al. (2003) Nat Struct Biol 10(9): 764-9.) Increased cell migration induced in NMuMg and malignant breast cancer cell lines by TGFβ is blocked by p38 MAPK inhibitor (Cohen, P. (2001) Eur J Biochem 268(19): 5001-10; Cuenda, A., et al. (1995) FEBS Lett 364(2): 229-33.) An inhibitor of the RhoA kinase, Y27632, blocks the formation of stress actin fibers and the migration of cells (Breitenlechner, C., et al. (2003) Structure (Camb) 11(12): 1595-607; ltoh, K., et al. (1999) Nat Med 5(2): 221-5; Roovers, K. and R. K. Assoian (2003) Mol Cell Biol 23(12): 4283-94.)

Needed in the art is an improved method of reversing epithelial mesenchymal cell transition.

### BRIEF SUMMARY OF THE INVENTION

As described above, the use of chemical inhibitors has been important in establishing several signaling pathways that are required for cells to undergo EMT, but much less is known about how the mesenchymal state is maintained or whether it is possible to reverse the process and re-form the epithelial cell phenotype. In the renal tubular epithelium, the reversal of EMT to reform the tubular epithelium is important for normal wound healing of a damaged tubule. In order to identify what pathways might be involved in reversal of EMT in renal tubular epithelial cells, we examined the effect of five different kinase inhibitors on the mesenchymal phenotype in mouse renal tubular epithelial cells. To study responses of tubular epithelial cells in the absence of autocrine TGF-β1, we used primary mouse tubular epithelial cells that had been isolated from the renal cortex of TGF-β1 knockout mice (mTEC-KO cells) (Grande J P, et al. (2002) Exp Biol Med (Maywood) 227:171-181). Although partial reversal of EMT morphology and patterns of gene expression were obtained by single kinase inhibitors, full reversal of morphology and cadherin gene expression required a combination of SB431542 and Y27632, i.e., inhibition of both the TGF-β and RhoA kinase pathways. We conclude that maintenance of the mesenchymal state in renal tubular epithelial cells uses independent, sustained signaling by both TβRI and ROCK.

In one embodiment, the present invention is a method of reversing epithelial mesenchymal transition, comprising the step of treating a fibrotic disease patient or cancer disease patient with an amount of kinase inhibitor capable of reversing EMT, wherein the kinase inhibitor comprises a TGF-βI kinase inhibitor and a Rho kinase inhibitor or a TGF-βI inhibitor and a p38 MAPK inhibitor.

In one embodiment, the administration of the inhibitors is simultaneous. In a preferred embodiment, the TGF-βI inhibitor is SB431542. In another preferred embodiment, the Rho kinase inhibitor is Y27632.

In one preferred embodiment, the p38 MAPK inhibitor is selected from the group consisting of SB203580 and SB202190.

In one preferred embodiment, the invention is a pharmaceutical composition comprising an amount of kinase inhibitor capable of reversing EMT, wherein the kinase inhibitor comprises a TGF-βI kinase inhibitor and a Rho kinase inhibitor or a TGF-βI inhibitor and a p38 MAPK inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: TGF-β1 induces EMT in renal tubular epithelial cells. mTEC-KOs were incubated for 72 hours without TGF-β1 (A,D), with 100 pM TGF-β1 (B, E) or with TGF-β1 and 10 μM SB431542. Cell morphology was observed by brightfield phase microscopy at 100× magnification (A-C). Phalloidin staining was observed at 400× magnification (D-F). White arrows point to stress fibers.

FIG. 2: TGF-β1 treatment of renal tubular epithelial cells reduces epithelial cadherin expression and increases mesenchymal marker gene expression. mTEC-KOs were incubated with 100 pM TGF-β1 for the indicated times and cells-were harvested for analysis of protein expression by Western blot with antibodies against E-cadherin, αSMA, and β-tubulin (A). mRNA expression in cell lysates was evaluated by quantitative RT-PCR for Ksp-cadherin (B), MMP-9 (C), and SM22 (D). Significant differences between cells without TGF-P treatment for 72 hours versus cells treated with TGF-β for the indicated times are indicated with an asterisk (*) (P<0.05, n=9).

FIG. 3: SB431542 reverts PAI-1 mRNA expression levels in TGFβ-induced mesenchymal renal tubular epithelial cells to levels comparable to epithelial cells. TGFβ1 ligand (100 pM) was added to mTEC-KOs for 72 hours, followed by the addition of 5 μM SB431542 plus 100 pM TGFβ1 for an additional time 24 hours. Cell lysates were used to prepare RNA which was examined by quantitative RT-PCR for PAI-1. Significant differences in PAI-1 expression level between cells treated with TGF-β1 alone versus cells treated with TGF-β1 and then with inhibitor for the indicated times are indicated with an asterisk (*) (P<0.05, n=9).

FIG. 4: Single kinase inhibitors fail to reverse the mesenchymal actin cytoskeleton induced in renal tubular epithelial cells but the combination of a TβRI inhibitor and a ROCK inhibitor eliminate detectable stress fibers. Renal tubular epithelial cells (mTEC-KOs) were treated with 100 pm TGF-β1 for 72 hours and then kinase inhibitors were added for an additional 24 hours. The F-actin in the cells was visualized by staining with phalloidin. mTEC-KO cells were treated with a single kinase inhibitor (A-E) or with SB431542 plus a second kinase inhibitor (F-I). Single kinase inhibitors and concentrations were: 5 μM SB431542 (A), 1 μM SB203580 (B), 1 μM Y27632 (C), 10 μM U0126 (D), or 15 μM SP600125 (E). Combination kinase inhibitors included 5 μM SB431542 with 1 μM SB203580 (F), 1 μM Y27632 (G), 10 μM U0126 (H) or 15 μM SP600125 (I). (J) Combination of RhoA kinase inhibitor and p38 MAPK inhibitor (5 μM Y27632 and 5 μM SB203580) did not alter the mesenchymal actin cytoskeleton. White arrows point to stress fibers.

FIG. 5: Restoration of epithelial gene expression patterns by kinase inhibitors illustrates a requirement for two kinase inhibitors to restore cadherin expression. mTEC-KO cells were treated with 100 pm TGF-β1 for 72 hours to induce EMT. Single kinase inhibitors or inhibitor combinations were added, cells were grown for an additional 24 hours and harvested for preparation of RNA. Ksp-cadherin (A), SM22 (B), and MMP-9 (C) mRNA levels were measured by quanitative RT-PCR. Significant differences between the untreated (No TGF-β1) cells versus cells treated with TGF-β1 or with TGF-β1 followed by inhibitor are indicated with an asterisk (*) (P<0.05, n=9). Significant differences between single inhibitors versus combination inhibitors are indicated by a letter (a,b,c) over the bar showing the combination. Each letter refers to the single inhibitor in the graph (indicated by the letter below the name of the inhibitor).

FIG. 6: E-cadherin is restored by combining TβRI kinase inhibitor with either the ROCK inhibitor or the p38 MAPK inhibitor. E-cadherin is found between mTEC-KO cells (A). mTEC-KO cells were treated with 100 pM TGF-β1 for 72 hours to induce mesenchymal state in which E-cadherin is lost (B). Single inhibitors or combinations of two inhibitors were added for an additional 36 hours. Single kinase inhibitors 5 μM SB431542 (C), 5 μM SB203580 (D), 5 μM Y27632 (E) slightly reformed E-cadherin while 15 μM SP600125.(E) did not express E-cadherin. E-cadherin reformed between cells that used combination kinase inhibitors which included 5 μM SB431542 with 5 μM SB203580 (G), 5 μM Y27632 (H), but not 15 μM SP600125 (I). Cell lysates from mTEC-KOs were analyzed by western blot using antibodies to E-cadherin and β-tubulin (J). Control epithelial cells were grown without TGF-β (Lane 3). The cells were then treated for an additional 48 hours with no inhibitor (Lane 2), single inhibitors (5 μM Y27632, Lane 6; 5 μM SB203580, Lane 7; 5 μM SB431542, Lane 8) or with the combination of 5 μM SB431542 with either 15 μM SP600125 (Lane 1), 5 μM Y27632 (Lane 4) or 5 μM SB203580 (Lane 5).

### DETAILED DESCRIPTION OF THE INVENTION

In General

Epithelial mesenchymal transition (EMT) is associated with the invasive behavior of metastatic cancers and with the pathological fibrosis that leads to organ failure, e.g. renal failure in end stage renal disease. Kalluri and Neilson (The Journal of Clinical Investigation Vol. 112, No. 12, December 2003, 1776-1784) describe epithelial mesenchymal transitions and their implications for fibrosis. Although single pharmacological inhibitors can block EMT, no pharmacological agents have been reported that reverse the process once it occurs. Single agents only reverse some of the molecular changes that occur in EMT.

In the Examples below, we have identified combinations of pharmacological inhibitors that cause reversal of EMT in mammalian cells. By combining two inhibitors for different pathways (targets), we find that EMT can be fully reversed. Such combinations of inhibitors will be useful in reversing organ damage caused by fibrosis and/or in treatment of metastic cancers.

Reversal of organ damage would best be indicated by return of normal organ function, which could include normal urine and blood chemistry for kidney, or normal lung function. The grading and staging of fibrosis in different tissues has been evaluated by serum markers, Doppler ultrasonography, CT and/or MR (magnetic resonance) imaging. The combination of these noninvasive parameters is sensitive and specific in the diagnosis of fibrosis. Reversal could also be evaluated in tissue biopsy by histological markers such as reduced myofibroblasts expressing smooth muscle actin, absence of fibrotic scarring and normal amounts of extracellular matrix proteins but noninvasive methods such as imaging would be preferred. Since the EMT derived myofibroblasts also activate tumor growth, reduction in tumor size would also be a measure as determined by standard of care imaging methods.

In a preferred version of the present invention, the pharmacological inhibitors are a combination of TGF-β Type I kinase inhibitor combined with Rho kinase inhibitor or a combination of TGF-β Type I kinase inhibitor with a p38 MAPK inhibitor.

Kinase Inhibitors

The present invention requires the combination of two kinase inhibitors. One may select a TGF-βI kinase inhibitor and a Rho kinase inhibitor or a TGF-βI kinase and a p38 MAPK inhibitor.

(a) TGF-βI Kinase Inhibitor

Transforming growth factor β-1 (TGF-βI) is known to induce transformations of cell morphology, motility and interactions with neighboring cells and exerts its signaling influence by activating a heteromeric receptor of two transmembrane serine/threonine kinases, Type I and Type II receptor kinases. Yingling, et al. (Nature Reviews, Drug Discovery [3] December, 2004, 1011-1022, incorporated by references herein) describes the two different receptor kinases. By “TGF-βI kinase inhibitor” we mean inhibitors of the R1 kinase activity.

The disclosure below demonstrates one particular TGF-βI kinase inhibitor, SB431542, as especially useful. Laping, et al., Molecular Pharmacology Vol. 62, No. 1 2002, 58-64; Matsuyama, et al., Cancer Research Vol. 63, November 15, 2003 7791-7798; Hjelmeland, et al., Mol Cancer Ther. Vol. 3, No. 6 June 2004 737-745 describe activity and characteristics of SB431452. SB431452, may be obtained commercially through Tocris, Cookson Inc., Ellisville, Mo. or Sigma-Aldrich, St. Louis, Mo.

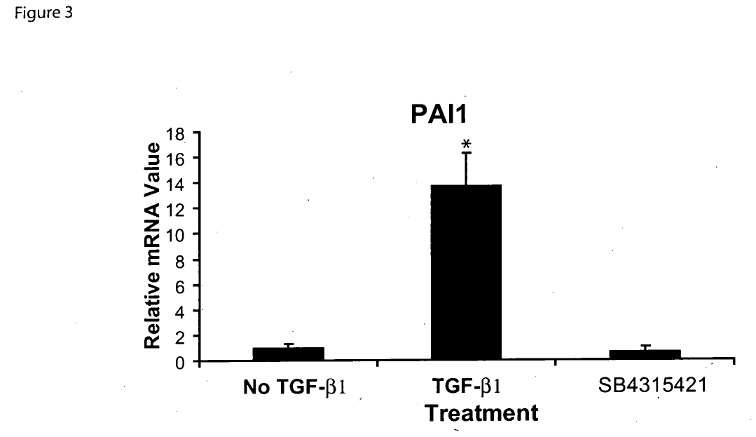

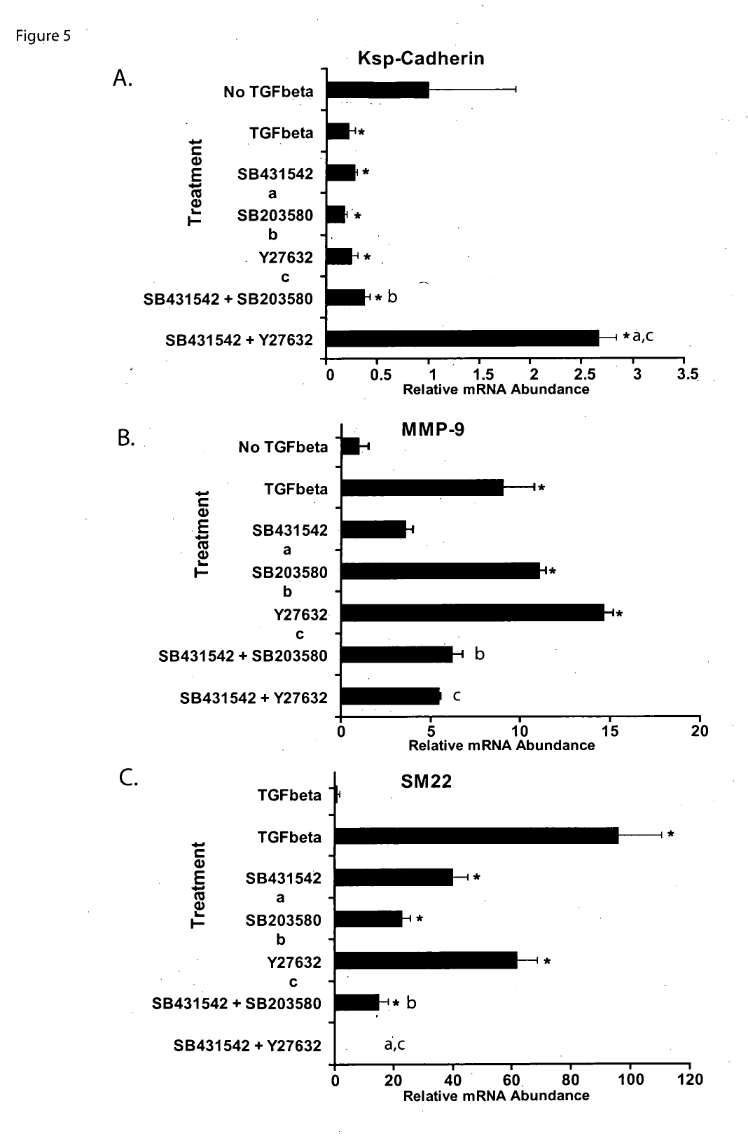

However, other TGF-βI inhibitors are known. For example, Yingling, et al. (incorporated by reference) lists numerous suitable TGF-β signaling inhibitors. Yingling, et al. also lists (see FIG. 3) many small molecule TGF-βR1 kinases inhibitors and notes that many individual molecule scaffolds have been developed as small molecule receptor kinase inhibitors. We mean to include any of the small molecule inhibitors and any other inhibitors that may significantly inhibit TGF-βR1 kinase. Yingling, et al. (supra) contains a description of testing for TGF-βR1 inhibitors. For example, see FIG. 5.

Specifically, we also mean to include the following inhibitors: SD-208, SB-525334, SM16, and LY21577299. Inhibitors are disclosed in Yingling, et al. (supra) or as follows:
Keystone Symposia Conference (Roles of TGF-β in Disease Pathogenesis: Novel Theraputic Strategies)
SD-208:
Guise, T. Role of TGF-β in Osteolytic Metastases of Breast Cancer, Roles of TGF-β in Disease Pathogensis, Keystone, Colo.; Keystone Symposia: Silverthorne, Colo., 2005; Abstract 012
SB-525334:
Laping, N. J., et al., ALK5 Kinase Inhibitors as Potential Treatment for Fibrosis and Uterine Fibrosis, Keystone, Colo.; Keystone Symposia: Silverthorne, Colo., 2005; Abstract 027
SM16 (Biogen):
Ling, L. E., et al., Identification by Virtual Screen and Potent Activity in Animal Models of Fibrosis, Keystone, Colo.; Keystone Symposia: Silverthorne, Colo., 2005; Abstract 214
American Association for Cancer Research (MCR) Special Conferences in Cancer Research (TGF-β in Cancer and Other Diseases)
LY2157299
Yingling, J. M., et al., A Clinical TGF-βRI Kinase Inhibitor for Cancer Therapy, La Jolla, Calif.; MCR Special Conference in Cancer Research: Philadelphia, Pa., 2006; Abstract Speaker
   (b) Rho Kinase Inhibitors and p38 MAPK Inhibitors

The EMT process is mediated by TGF-βI induced activation of protein complexes but also requires cooperation of multiple other cellular signaling factors such as RhoA and p38 mitogen activated protein kinase (p38 MAPK). A number of small molecule drugs have recently been discovered that are specific to these pathways. For example, Y27632 is a known inhibitor of Rho kinase and SB203580 and SB202190 are chemical inhibitors of p38 MAPK and are in clinical trials as anti-inflammatory agents. SCIO-469 (Hideshima, T., et al. (2004) Oncogene 23(54): 8766-76; Nikas, S. N. and A. A. Drosos (2004) Curr Opin Investig Drugs 5(11): 1205-12) is another preferred p38 MAPK inhibitor.

Tada, et al. (J. Hepatol. Vol. 34, No. 4, April 2001, 529-536) describes Y27632. One may most easily obtain Y27632 from Calbiochem, San Diego, Calif. and Sigma-Aldrich, St. Louis, Mo.

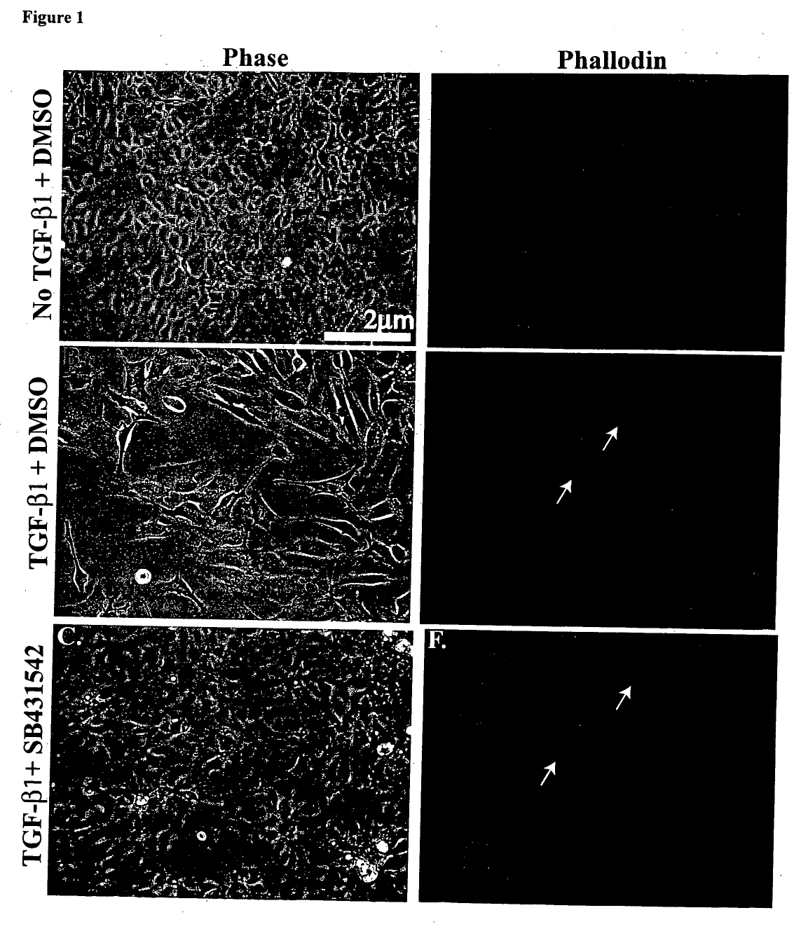

Breitenlechner, et al. (Structure Vol. 11, December 2003, 1595-1607, incorporated by reference) describes several Rho kinases inhibitors and a structural basis for their selectivity. For example, see FIG. 1, Breitenlechner, et al. We mean for the inhibitors described in Breitenlechner, et al. to be useful in the present invention and other Rho kinase inhibitors that may be known or discovered.

We note that a different approach to inhibiting the Rho pathway may be via statins. Two sets of scientists, Porter, et al. and Watts and Spiteri (Porter, et al., Cardiovasc. Res. 61(4):745-755, 2004; Watts and Spiteri, Am. J. Physiol. Lung Cell Mol. Physiol. 287:L1323-L1332, 2004, both incorporated by reference) have demonstrated the inhibition of RhoA via simvastatin. We mean to specifically include statin inhibition of Rho kinase in an alternative embodiment of the present invention. For example, Pravastatin (Casani, L., et al. (2005) Thromb Haemost 94(5): 1035-41), Fluvastatin (Shibata, S., et al. (2006) J Am Soc Nephrol 17(3): 754-64), Lovastatin (Lee, J., et al. (2006) Biochem Biophys Res Commun 339(3): 748-54), Cerivastatin (Takeuchi, S., et al. (2000) Biochem Biophys Res Commun 269(1): 97-102) are suitable.

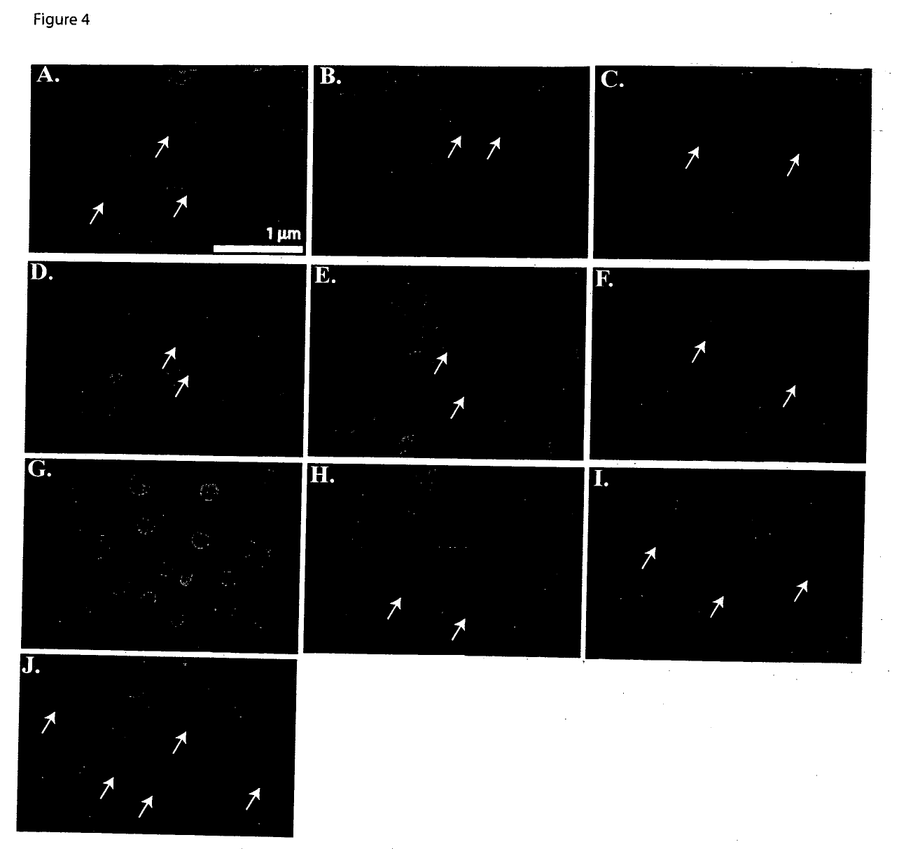

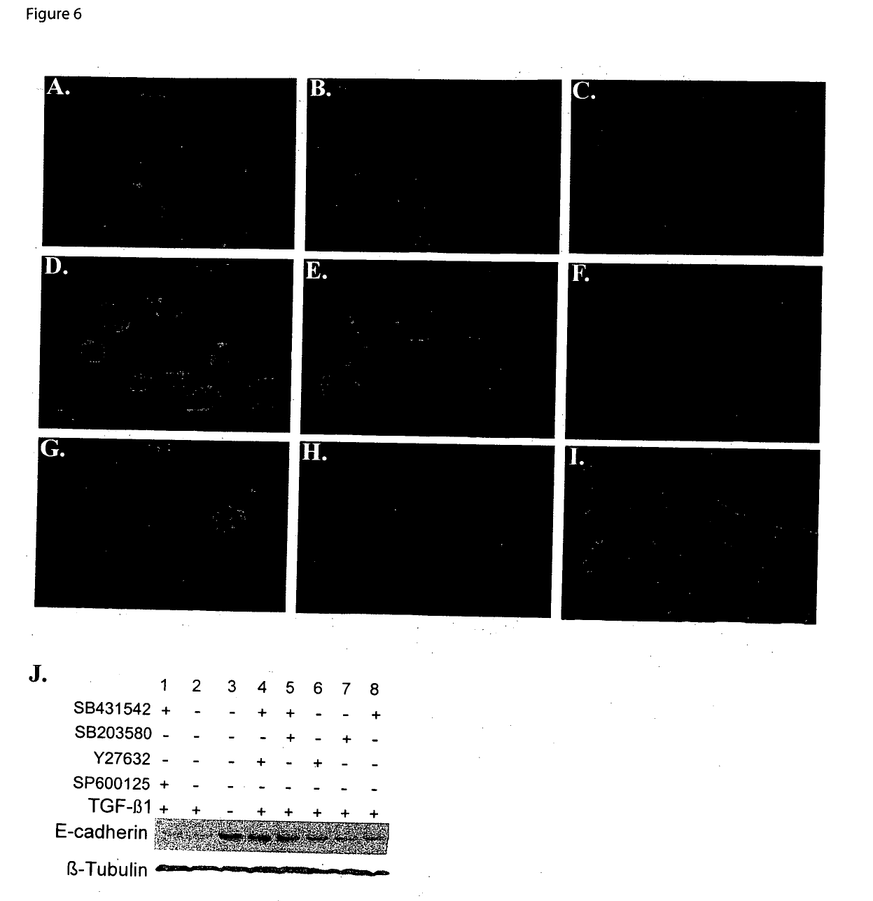

Kumar, et al. (Nature Reviews Vol. 2, September 2003, 717-726, incorporated by reference) describes various p38MAP kinases. For example, FIG. 4 of Kumar, et al. describes structures of representative classes of p38MAP kinase inhibitors. FIG. 6 describes structures of p38MAP kinases inhibitors that have advanced to clinical trials. We mean for the inhibitors described in Kumar, et al. to be useful in the present invention and any other p38MAP kinase inhibitors that may be known or discovered. Kumar, et al. contains information sufficient to determine whether a molecule is a p38 MAP kinase inhibitor.

p38 MAPK inhibitors that are especially useful for the present invention, SB203580 and SB202190, are most easily commercially available from Calbiochem, Tocris, Cookson Inc., Ellisville, Mo. or Sigma-Aldrich, St. Louis, Mo.

Prophetic Treatment Method

The present invention will be useful for treating a number of diseases associated with EMT. We mean for these to especially include fibrotic diseases such as diabetic nephropathy, liver cirrhosis, scleroderma, scarring, keloids and adhesions. We also mean to include cancer patients.

One could use the inhibitors of the present invention in similar doses and administration as they are currently used in treatment of disease. For example, Yingling, et al. (supra) lists references to appropriate clinical doses (see, for example, Table 1.) However, one would use a combination of inhibitors as described above. According to the method of the present invention, one would use an amount of inhibitors effective to reverse EMT.

We envision that one would typically use the inhibitors of the present invention in a pharmacological composition designed for most effective treatment. By “pharmacological composition,” we mean that the composition, in addition to nontoxic, inert pharmaceutically suitable excipients, contains one or more active compounds according to the invention. Preferably, the pharmaceutical composition comprises a combination of inhibitors as described above. The pharmaceutical preparations of the invention can also contain further pharmaceutical active compounds in addition to the active compounds according to the invention.

Nontoxic inert pharmaceutically suitable excipients are understood as meaning pharmaceutically acceptable solid, semisolid or liquid diluents, fillers and formulation auxiliaries of any type, which after mixing with the active compound bring this into a form suitable for administration. Suitable administration forms of the compounds according to the invention are, for example, tablets, coated tablets, capsules, pills, aqueous solution, suspensions and emulsions, if appropriate sterile injectable solutions, nonaqueous emulsions, suspensions and solutions, sprays and also preparation forms with protracted release of active compound.

The inhibitors described above can be incorporated into standard pharmaceutical dosage forms, for example, for oral, topical or parenteral application with the usual pharmaceutically acceptable adjuvant materials, for example, organic or inorganic inert carrier materials, such as, water, gelatin, lactose, starch, magnesium stearate, talc, vegetable oils, gums, polyalkylene-glycols and the like. The pharmaceutical compositions can be employed in a solid form, for example, as tablets, suppositories, capsules, or in liquid form, for example, as solutions, suspensions or emulsions. Pharmaceutically acceptable adjuvant materials can be added and include preservatives stabilizers, wetting or emulsifying agents, salts to change the osmotic pressure or to act as buffers. The pharmaceutical compositions can also contain other therapeutically active substances.

The dosage can vary within wide limits and will, of course, be fitted to the individual requirements in each particular case. In the case of oral administration, the dosage lies in the range of about 0.1 mg per dosage to about 1200 mg per day of the inhibitors described above, although the upper limit can also be exceeded when this is shown to be indicated. Preferably, a patent would receive 4 mg-600 mg a day. The inhibitor combination is preferably administered simultaneously, although in other embodiments of the invention, the inhibitors may be administered sequentially.

The following references describe Rho kinase inhibitors in clinical use:
Shimokawa, et al., J. Cardiovasc. Pharmacol. 40(5):751-761, 2002; Department of Cardiovascular Medicine, Kyushu University Graduate School of Medical Sciences, Fukuoka, Japan. In this multicenter phase II study, the anti-anginal effect of fasudil, which is metabolized to a specific Rho-kinase inhibitor hydroxyfasudil after oral administration, was examined in patients with stable effort angina.
Shibuya, et al., J. Neurosurg. 76(4):571-577, 1992; Department of Neurosurgery, Nagoya University, Japan. With the cooperation of 60 neurosurgical centers in Japan, a prospective randomized placebo-controlled double-blind trial of a new calcium antagonist AT877 (hexahydro-1-(5-isoquinolinesulfonyl)-1H-1,4-diazepine hydrochloride, or fasudil hydrochloride) was undertaken to determine the drug's effect on delayed cerebral vasospasm in patients with a ruptured cerebral aneurysm.

Palladino, et al. (Nat. Rev. Drug Discov. 2(9):736-746, 2003) summarizes P38 inhibitors in current clinical use (incorporated by reference as if fully set forth herein).

Hasegawa, et al., (J Dermatol Sci. 39(1):33-8, July 2005), describes the use of SB431542 in suppression of keloid fibroblasts.

One may wish to use the articles described below to evaluate the efficacy of compounds and the efficacy of EMT reversal. We mean for reversal of EMT to comprise a significant clinical change in primary disease parameters. For example, Rosenberg, et al. describes serum markers to detect the presence of liver fibrosis. A good clinical method is to look for normal function of the kidney, lung or other affected organ. If one wished to reverse the disease, many of the serum markers that are associated with the disease would decrease.

| Authors | Article |
|---|---|
| Rosenberg, W. M., | Serum Markers Detect the Presence of Liver |
| et al. | Fibrosis: A Cohort Study, Gastroenterology 127 |
| | (6): 1704-1713, 2004. |
| Poynard, T., | Biochemical Surrogate Markers of Liver Fibrosis |
| et al. | and Activity in a Randomized Trial of |
| | Peginterferon Alfa-2b and Ribavirin, Viral |
| | Hepatology 38(2): 481-492, 2003. |
| Zeisberg, M., | BMP-7 Counteracts TGF-β1-induced |
| et al. | Epithelial-to-mesenchymal Transition and |
| | Reverses Chronic Renal Injury, Nature Medicine |
| | 9(7): 964-968, 2003. |
| Barnes, P. J. | Prospects for New Drugs for Chronic Obstructive |
| and | Pulmonary Disease, The Lancet 364: 985-996, |
| T. T. Hansel | 2004. |
| Bissell, D. M. | Assessing Fibrosis Without a Liver Biopsy: Are |
| | We There Yet?, Gastroenterology 1127(6): |
| | 1847-1849, 2004. |
| Ciulla, M. M., | Different Effects of Antihypertensive Therapies |
| et al. | Based on Losartan or Atenolol on Ultrasound and |
| | Biochemical Markers of Myocardial Fibrosis: |
| | Results of a Randomized Trial, Circulation |
| | 110(5): 552-557, 2004. |
| El-Agroudy, | Effect of Angiotensin II Reeptor Blocker on |
| A. E., et al. | Plasma Levels of TGF-beta 1 and Interstitial |
| | Fibrosis in Hypertensive Kidney Transplant |
| | Patients, Am. J. Nephrol. 23(5): 300-306, |
| | 2003. |
| Amann, B., | ACE Inhibitors Improve Diabetic Nephropathy |
| et al. | Through Suppression of Renal MCP-1, Diabetes |
| | Care 26(8): 2421-2425, 2003. |
| Padi, S. S. | Salvage of Cyclosporine A-induced Oxidative |
| and | Stress and Renal Dysfunction by Carvedilol, |
| K. Chopra | Nephron. 92(3): 685-692, 2002. |
| Padi, S. S. | Selective Angiotensin II Type 1 Receptor |
| and | Blockade Ameliorates Cyclosporine |
| K. Chopra | Nephrotoxicity, Pharmacol. Res. 45(5): |
| | 413-420, 2002. |
| Kobayashi, N., | Effects of Oral Adsorbent AST-120 (Kremezin) |
| et al. | on Renal Function and Glomerular Injury in |
| | Early-stage Renal Failure of Subtotal |
| | Nephrectomized Rats, Nephron. 91(3): |
| | 480-485, 2002. |
| Man, J. and | Use of Immiquimod Cream 5% in the Treatment of |
| M. T. Dytoc | Localized Morphea, J. Cutan. Med. Surg. 8(3): |
| | 166-169, 2004. |
| Mancuso, G. | Topical Tacrolimus in the Treatment of |
| and R. M. | Localized Scleroderma, Eur. J. Dermatol. 13(6): |
| Berdondini | 590-592, 2003. |
| Ling, T. C., | Keloidal Scleroderma, Clin. Exp. Dermatol. |
| et al. | 28(2): 171-173, 2003. |
| McGaha, T. L, | Halofuginon Inhibition of COL1A2 Promoter |
| et al. | Activity via a c-Jun-dependent Mechanism, |
| | Arthritis Rheum. 46(10): 2748-2761, 2002. |
| Currie, D. M., | Topical Treatment of Sclerodermoid Chronic |
| et al. | Graft vs. Host Disease, Am. J. Phys. Med. |
| | Rehabil. 81(2): 143-149, 2002. |

The following references describe medical imaging as an evaluation of fibrosis and should be consulted if one wishes to use medical imaging to evaluate EMT reversal.

| Authors | Article |
|---|---|
| Robinson, T. E. | High-resolution CT Scanning: Potential Outcome |
| | Measure, Curr. Opin. Pulm. Med. 10(6): 537-541, |
| | 2004. |
| Weitzel, W. F., | Feasibility of Applying Ultrasound Strain |
| et al. | Imaging to Detect Renal Transplant Chronic |
| | Allograft Nephropathy, Kidney Int. 65(2): 733- |
| | 736, 2004. |
| Konen, E., et al. | Fibrosis of the Upper Lobes: A newly Identified |
| | Late-onset Complication After Lung |
| | Transplantation?, AJR Am. J. Roengenol. 181(6): |
| | 1539-1543, 2003. |
| Afdhal, N. H. | Evaluation of Liver Fibrosis: A Concise Review, |
| and D. Nunes | Am. J. Gastroenterol. 99(6): 1160-1174, 2004. |
| Lu, L. G., | Grading and Staging of Hepatic Fibrosis, and its |
| et al. | Relationship with Noninvasive Diagnostic |
| | Parameters, World J. Gastroenterol. 9(11): 2574- |
| | 2578, 2003. |

One may wish to include multiple TGF-βI kinase inhibitors, Rho kinase inhibitors or MAPK kinase inhibitors. For example, one may wish to combine several TGF-β Type I kinase inhibitors with a Rho kinase inhibitor or several Rho kinase inhibitors with a TGF-β Type I kinase inhibitor.

### EXAMPLE

I. Reversal of Epithelial to Mesenchymal Transition in Tubular Epithelial Cells by Combining Two Kinase Inhibitors

A. In General

Epithelial to mesenchymal transition (EMT) is a normal developmental process that is also associated with the progression of cancer and with fibrotic diseases such as diabetic nephropathy. EMT can be blocked by inhibition of a number of cell signaling components, however, there are few reports of reversing EMT once the mesenchymal state is established. Inhibitors of five kinases implicated in EMT, transforming growth factor-beta (TGF-β) Type I receptor kinase, p38 mitogen-activated protein kinase (p38 MAPK), MAPK/extracellular signal-regulated kinase, c-Jun NH-terminal kinase and RhoA kinase, were evaluated for reversal of the mesenchymal state induced in renal tubular epithelial cells. None of these was able to fully restore epithelial morphology or gene expression as single agents. In contrast, exposure to the TGF-β Type I receptor kinase inhibitor, SB431542, combined with the RhoA kinase inhibitor, Y27632, eliminated detectable actin stress fibers and mesenchymal gene expression while restoring epithelial E-cadherin and kidney specific cadherin expression. A second combination, SB431542 with the p38 MAPK inhibitor, SB203580, was partially effective in reversing EMT. Our results indicate Rho Kinase or p38 MAPK act with TGF-β Type I receptor kinase to maintain the mesenchymal phenotype.

B. Materials and Methods

Reagents:

TGF-β1 was from R&D Systems (Minneapolis, Minn.); TGF-β1 knockout Murine Renal Tubular Epithelial Cells were generously provided to Dr. Bryan Becker at UW-Madison by Dr. Jeffrey Kopp (National Institute of Diabetes and Digestive and Kidney Diseases, MD). Chemical inhibitors were obtained from Calbiochem (SB431542, SB203580, SP600125), Tocris (Y27632) or Promega (U0126).

Cell Culture

TGF-β knockout Murine Renal Epithelial Tubular Cells (mTEC-KO) were maintained in Renal Epithelial Cell Growth Medium (Cambrex, Md.) containing 0.25% Fetal Bovine serum (FBS), supplemented with a Bullet Kit that contained: epidermal growth factor, insulin, hydrocortisone, GA-1000, epinephrine, T₃, transferrin (Cambrex), as well as penicillin and streptomycin (Gibco) in a 37° humidified 5% CO₂ incubator.

Western Blot Analysis and Antibodies:

In a P100 plate, 100,000 cells were seeded and appropriately treated. Cells were washed with cold PBS, lysed in TNE buffer (50 mM Tris-HCl (pH 8.0), 1% NP40, 150 mM NaCl, 5 mM EDTA) and centrifuged for 5 min at 4° C. Lysates prepared using TNE buffer were supplemented with protease inhibitor cocktail tablet (Roche). Cell homogenates were incubated for 10 min at 100° C. in loading buffer. Equal amounts of protein were added to each well, as assessed by BCA Protein Assay Kit (Pierce, Ill.) and loaded onto 4-20% polyacrylamide gels (ISC BioExpress, Utah), separated by electrophoresis, and transferred to PVDF membrane (Millipore, Mass.). The antibodies used for detection were: E-cadherin (BD Biosciences), β-tubulin (Sigma), and α-Smooth Muscle Actin (Sigma). Anti-mouse IgG conjugated with horseradish peroxidase (Santa Cruz) was used as the secondary antibody. Blots were developed by using ECL (Amersham Biosciences). The blots were stripped by incubating with 100 mM β-mercaptoethanol/2% SDS/62.5 mM Tris (pH 8.2) at 65° C. for 1 hour and reprobed with primary antibody and HRP secondary.

Quantitative PCR:

In a six well plate, 50,000 cells were seeded and appropriately treated. Total RNA was isolated using RNAeasy Miniprep kit (Qiagen) and quantified by UV spectrophotometer. 1.5 μg of RNA was converted by reverse transcriptase into cDNA using the OmniScript kit (Qiagen). Primers used for Q-PCR were: KSP-Cadherin forward-5′ CTG CAC ACA GM GTC CCT GA 3′, reverse 5′ CCT TGT CGC CAC TAG AAA GC 3′; MMP-9-SuperArray (Frederick, Md.) PPM03661A; SM22 forward 5′GCA GTC CM MT TGA GM GA 3′, reverse 5′ CTG TTG CTG CCC ATT TGA AG 3′; PAI-1 forward 5′TTCAGCCCTTGCTTGCCTC 3′, reverse 5′ACACTTTTACTCCGMGTCGGT 3′. cDNA was amplified in an Opticon 2 PCR machine (MJ Research) and labeled by the DyNAmo SYBR Green qPCR Kit. The amplification was carried out in the following manner: initial denaturation for 10 min at 95° C., denaturation for 10 sec at 95° C., annealing for 30 sec at the appropriate temperature, and extension for 30 sec at 72° C. Standards were created from Pfu (Stratagene) amplified PCR products purified by gel purification. Glyceraldehyde-3-phosphate dehydrogenase (GAPDH) was used as an internal control to normalize gene expression. The primer sequences for mouse GAPDH were Forward: 5′ AGG TCG GTG TGA ACG GAT TTG 3′ and Reverse: 5′ TGT AGA CCA TGT AGTTGA GGT CA 3′.

Immunofluorescence:

Cells were seeded on glass cover slips (Fisher) and cultured as described above. Cells were treated, fixed in 4% paraformaldehyde, and permeabilized in PBS containing 0.1% Triton X-100 for 10 min. Nonspecific sites were blocked with 10% BSA or normal goat serum for 30 min. F-actin was stained at a 1:1000 dilution in 2% BSA of Texas Red-conjugated phalloidin (Sigma) and incubated for 30 min. E-cadherin (BD Biosciences) was diluted at 1:200 in 2% normal goat serum and a secondary antibody (Molecular Probes) at a 1:500 dilution was used for detection. All washes were done in 1× PBS.

Statistical Analyses:

All experiments were performed at least twice, each time with individual duplicates. Figures show the mean of the triplicates for one representative experiment but for the statistical evaluation, the data from all experiment replicates were pooled. For comparisons between pairs, we used the one-way analysis of Wilcoxson Rank Sum using the MSTAT software (http:/Imcardle.oncology.wisc.edu/mstat). A p value <0.05 was considered statistically significant.

C. Results

In the absence of TGF-β1, mTEC-KO cells form an epithelial sheet resembling a cobblestone pattern (FIG. 1A). Addition of 100 μM TGF-β1 for 72 hours induced the mTEC-KO cells to acquire a spindle shape and a more fibroblast-like morphology (FIG. 1B). The morphological transformation correlated with major changes in the actin cytoskeleton as revealed by phalloidin staining (FIG. 1D, E). Untreated epithelial cells exhibited a cortical actin stain below the cell membranes associated with cell-cell junctions whereas the TGF-β1 treated cells displayed actin stress fibers, defined here as elongated F-actin spanning the length of the cell. The TβRI inhibitor SB431542 blocked the TGF-β1-induced transition of the mTEC-KO epithelial cells into mesenchymal cells (FIG. 1C). However, some non-cortical actin fibers, defined here as short phalloidin-stained fibers that are not at the cell junctions, were detectable in the cells, though very few actin stress fibers were observed (FIG. 1F).

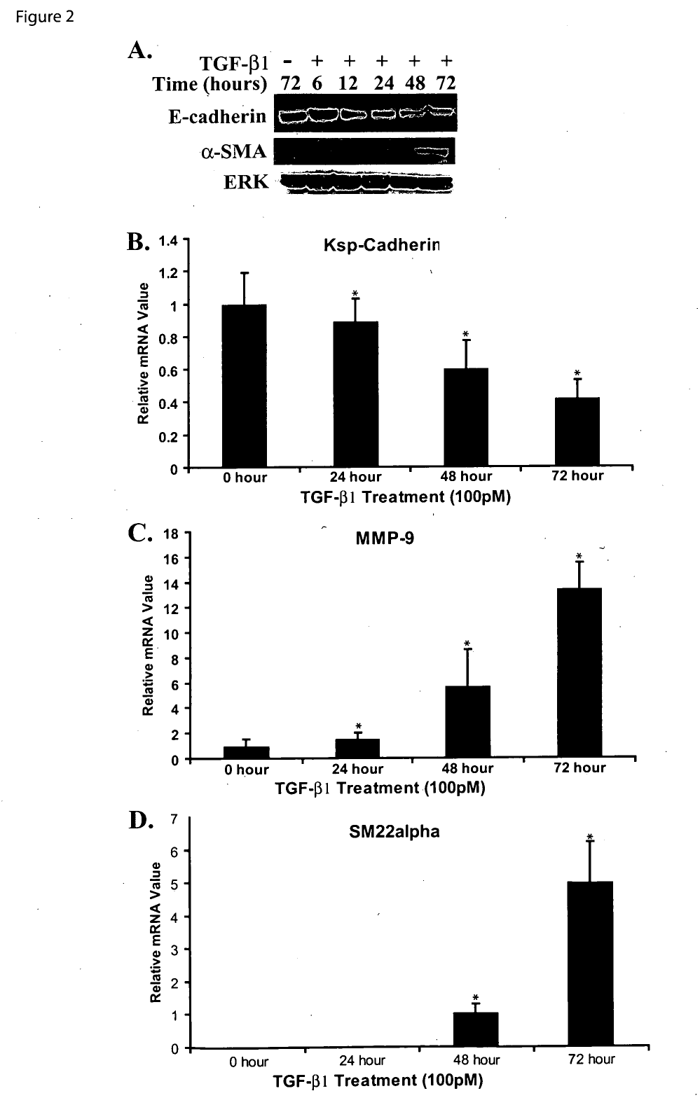

The structural integrity and polarization of the epithelial cells is maintained by E-cadherins binding to a network of actin filaments; the reduction of E-cadherin expression is a hallmark of mesenchymal acquisition (Shook D and Keller R (2003) Mech Dev 120:1351-1383). We examined the expression levels of a number of genes regulated by TGF-β1 as markers for the epithelial and mesenchymal cell state (FIG. 2). In mTEC-KO cells, addition of TGF-β1 decreased expression of the epithelial protein E-cadherin and induced expression of the mesenchymal protein a-smooth muscle actin (α-SMA) at 72 hours (FIG. 2A). Because TGF-β1 is known to regulate expression of multiple cadherins, we also examined expression of kidney specific cadherin (Ksp-cadherin). Ksp-cadherin has a similar developmental pattern of expression as the tight junction proteins ZO-1 and claudin-3 in kidney epithelial cells and therefore, is used as a marker of the epithelial state (Meyer T N, et al. (2004) Dev Biol 275:44-67). The addition of TGF-β1 reduced Ksp-cadherin mRNA expression (FIG. 2B), while increasing the mRNA levels of the mesenchymal markers matrix metalloproteinase-9 (MMP-9) (FIG. 2C), and the smooth muscle protein 22 (transgelin) (SM22) (FIG. 2D).

To examine the reversibility of the EMT induced by TGF-β1 in mTEC-KO cells, we added inhibitors of kinases previously implicated in EMT. Cells were treated with 100 pM TGF-β1 for 72 hours to induce EMT and kinase inhibitors were added for an additional 24 hours. Addition of TβRI inhibitor SB431542 at 5 uM for 24 hours was sufficient to reduce the mRNA level of the TGF-β-responsive gene plasminogen activator inhibitor-1 (PAI-1), demonstrating that TGF-β1 signaling was effectively inhibited (FIG. 3). To assess the effects of the kinase inhibitors on EMT, the actin cytoskeleton was examined by phalloidin staining. In contrast to its ability to prevent induction of EMT by TGF-β1 (FIG. 1) and reverse the elevated PAI-1 expression, SB431542 did not reverse the mesenchymal actin stress fiber morphology of the TGF-β1-treated mTEC-KO cells (FIG. 4A). Inhibition of other kinases previously implicated in inducing EMT, p38 MAPK, MEK1, JNK or ROCK also did not reverse the actin stress fiber morphology induced in the mTEC-KO cells by TGF-β1 (FIGS. 4B-E).

Since EMT effects are mediated by multiple cellular pathways and none of the single kinase inhibitors reverted the EMT phenotype, we applied combinations of two inhibitors. When 5 μM SB431542 was combined with either 1 μM SB203580 or 1 μM Y27632 for 24 hours, the epithelial appearance of the cells was restored (FIG. 4F and G). SB431542 and SB203580 (FIG. 4F) reduced the stress fibers in comparison to either single treatment alone; however non-cortical actin filaments were still detectable. Detectable actin stress fibers were eliminated by the combination of SB431542 and Y27632 (FIG. 4G). Cortical actin bordering the cell-cell junctions was restored by both combinations. The addition of either the MEK1 inhibitor or the JNK inhibitor to the T,RI inhibitor had no detectable effect on the mesenchymal phenotype of the cells (FIGS. 4H and I). The combination of SB203580 and Y27632 restored cortical actin staining, but stress fiber actin remained in the cells (FIG. 4J).

The effects of individual or combinations of kinase inhibitors on the expression of several genes altered by EMT were measured by quantitative RT-PCR. TGF-β1 treatment reduced Ksp-cadherin levels within 24 hours (FIG. 2). Treatment with either SB431542 or Y27632 increased Ksp-cadherin to intermediate levels, in between non-TGF-β1-treated epithelial cells and TGF-β1-induced mesenchymal cells (FIG. 5A). The p38 inhibitor SB203580 had the opposite effect and led to a further decrease in Ksp-cadherin expression. The combination of SB431542 and SB203580 was not statistically different from SB431542 alone, but the combination of SB431542 and Y27632 caused an increase in Ksp-cadherin levels that were significantly greater than the levels achieved by either inhibitor alone (FIG. 5A).

SB431542 efficiently reduced SM22 and MMP-9 expression to pre-EMT levels (FIGS. 5B and C). SB203580 did not reduce SM22 or MMP9 transcript levels, indicating that the p38 MAPK inhibitor was not able to reverse the expression of these genes that are associated with the mesenchymal state. Y27632 partially reduced SM22 levels (FIG. 5B) but increased the level of MMP9 expression (FIG. 5C). This increase in MMP9 expression level was overcome by the combined SB431542 and Y27632 treatment (FIG. 5C).

An important criterion for epithelium restoration is re-expression of the cell-cell junction adhesion protein E-cadherin. mTEC-KO cells were treated with 100pm TGF-β1 for 72 hours to induce EMT and then kinase inhibitors were added for an additional 24-48 hours (FIG. 6). The single inhibitors SB431542 (FIG. 6C), Y27632 (FIG. 6D) or SB203580 (FIG. 6E) caused an increase in reforming E-cadherin at cell junctions as compared to TGF-β1-treated-mTEC-KOs (FIG. 6B), but the combination of SB431542 with either SB203580 (FIG. 6G) or Y27632 (FIG. 6H) restored E-cadherin localization indistinguishable from that of non-TGF-β1-treated mTEC-KO cells (FIG. 6A). SP600125 (FIG. 6F) or a combination of SB431542 with SP600125 did not restore E-cadherin (FIG. 6I). The combination of TGFβRI and ROCK inhibitors also were most effective in restoring the protein level, as well as the localization of E-cadherin as determined by western blot analysis of cell lystates (FIG. 6J).

D. Discussion

A series of recent investigations demonstrated that EMT is involved in the pathogenesis of experimental, native and transplant kidney disease in both animals and humans (Rastaldi M P, et al. (2002) Kidney Int 62:137-146; Li Y, et al. (2003) J Clin Invest 112:503-516; Djamali A, et al. (2004) American Journal of Transplantation OnlineEarly doi: 10.1111 /j.1600-6143.2004.00713.x). In renal fibrosis, tubular epithelial cells undergo EMT in response to primary insults such as hypertension or diabetes, resulting in the production and deposition of excessive extracellular matrix proteins (Zeisberg M and Kalluri R (2004b) J Mol Med 82:175-181). Numerous studies have reported inhibiting the induction of EMT by interfering with various cellular pathways comprising TGF-β receptors, RhoA kinase, p38 MAPK, integrins and PI3K (Miettinen P J, et al. (1994) J Cell Biol 127:2021-2036; Portella G, et al. (1998) Cell Growth Differ 9:393404; Bakin A V, et al. (2000) J Biol Chem 275:36803-36810; Bhowmick N A, et al. (2001b) Mol Biol Cell 12:27-36; Bakin A V, et al. (2002) J Cell Sci 115:3193-3206).

Studies with small molecule inhibitors, dominant negative proteins or RNAi have examined the effect of those inhibitors when added before or coincident with the EMT stimulus. We report that none of five different kinase inhibitors were able-to reverse EMT in renal tubular epithelial cells as single agents, but that the combination of a TGF-β receptor inhibitor and a RhoA kinase inhibitor caused a dramatic reversal in 24 hours of cell morphology and gene expression. The combination of two kinase inhibitors eliminated actin stress fibers and restored cadherin expression and localization associated with the epithelial cell phenotype. These results provide the first indication that maintenance of the mesenchymal state in tubular epithelial cells involves sustained and independent signaling through these two kinases.

TGF-β signaling is most likely maintaining the mesenchymal state by sustained activation of transcriptional responses to the activated Smad proteins. TGF-β exerts its signaling effects by activating a heteromeric receptor of two transmembrane serine/threonine kinases, type I and type II receptors (TβRI and TβRII) (Derynck R and Zhang Y E (2003) Nature 425:577-584.) TβRII transphosphorylates and activates the kinase function of TβRI. TβRI directly phosphorylates the cytoplasmic proteins Smad2 and Smad3 at carboxy-terminal serines. The phosphorylated Smad2 and Smad3 associate with Smad4, translocate into the nucleus and interact with over two dozen identified transcription factors, coactivators, and corepressors to regulate gene expression (ten Dijke P and Hill C S (2004) Trends Biochem Sci 29:265-273.) The suppression of TGF-β1 either by neutralizing antibodies or chemical inhibitors ameliorates the progression of EMT, thereby blocking the damaging results of TGF-β1 in organ fibrosis (Bottinger E P and Bitzer M (2002) J Am Soc Nephrol 13:2600-2610; Dai C, Yang J and Liu Y (2003) J Biol Chem 278:12537-12545; Schnaper H W, et al. (2003) Am J Physiol Renal Physiol 284:F243-252). The RhoA kinase pathway may be responsible for maintaining the actin stress fibers in the mesenchymal cells through activation of LIM kinase and cofilin (Vardouli L, et al. (2005) J Biol Chem.) but may also have roles in regulating gene expression, perhaps through its reported effects on the Jak-Stat and NFkB pathways (Benitah S A, et al. (2004) Biochim Biophys Acta 1705:121-132.)

Renal tubular epithelial cells were chosen for these studies because of the correlation between the extent of tubulointerstitial fibrosis and the prognosis for end stage renal disease (Nath K A (1992) Am J Kidney Dis 20:1-17.) Tubular epithelial cells express endogenous TGFβ1 in response to several stimuli including angiotensin II, cyclosporin A, and high glucose (Rocco M V, et al. (1992) Kidney Int 41:107-114; Wolf G, et al. (1993) J Clin Invest 92:1366-1372; Johnson D W, et al. (1999) J Pharmacol Exp Ther289:535-542) and, in culture, autocrine TGFβ production by wildtype mouse tubular epithelial cells results in a concentration of active plus latent TGFβ1 of approximately 100 pM in the conditioned medium, where a significant fraction (<40 pM) is in the active form, enough to affect the growth and gene expression in the cells (Grande J P, et al. (2002) Exp Biol Med (Maywood) 227:171-181.) To study responses of tubular epithelial cells in the absence of autocrine TGFβ1, primary renal epithelial cells were isolated from the renal cortex of TGFβ1 knockout (KO) mice (Grande J P, et al. (2002) Exp Biol Med (Maywood) 227:171-181; Chen S, et al. (2004) Kidney Int 65:1191-1204.) Both the renal tubular epithelial cells from wild type mice and the TGFβ knockout tubular epithelial cell (mTEC-KO) populations expressed the epithelial markers cytokeratin and E-cadherin, were negative for the expression of the mesenchymal markers smooth muscle actin, desmin and vimentin.

Elevated glucose did not cause cellular hypertrophy or increased fibronectin expression in mTEC-KO cells, consistent with these responses being mediated by activation of autocrine TGFβ1 (Chen S, et al. (2004) Kidney Int 65:1191-1204). However, addition of exogenous TGFβ1 normalized the differences in cell growth and induced collagen IV and fibronectin expression, consistent with the presence of normal TGF-β1 responses in the mTEC-KO cells. Using these renal tubular epithelial cells, we found that partial reversal of the EMT morphology and patterns of gene expression were obtained by single kinase inhibitors, but that full reversal of the morphology and cadherin gene expression required a combination of SB431542 and Y27632, i.e., inhibition of both the TGFβ and RhoA kinase pathways. Similar results were demonstrated in wild type mTEC cells. The combination of TGFβRI and ROCK inhibitors reversed EMT as indicated by gene expression and cell morphology (data not shown).

There are a few reports of reversing the mesenchymal state to an epithelial phenotype using macromolecules such as BMP7, Hepatocyte Growth Factor (HGF), E-cadherin or dominant negative proteins rather than small molecule inhibitors. TGF-β1 induced EMT in mouse distal tubular epithelial cells was reversed by addition of 100 ng/ml BMP7 for 48 hours as indicated by restoration of E-cadherin expression and epithelial morphology (Zeisberg M, et al. (2003) Nat Med 9:964-968; Zeisberg M and Kalluri R (2004a) Blood Purif 22:440445.) BMP-7 can also induce mesenchymal to epithelial transition (MET) in adult kidney fibroblasts (Zeisberg M, et al. (2004) J Biol Chem.) When a mouse model of chronic renal injury induced by nephrotoxic serum was treated with 300 ug/kg of human BMP7 every other day beginning at week three, BMP-7 reversed the renal pathology as indicated by a decline in mortality, and improvement in renal function. BMP-7 is a TGFβ superfamily member that acts through distinct receptors and Smad proteins to regulate gene expression (Kalluri R and Zeisberg M (2003) Histol Histopathol 18:217-224) but it is currently not clear how BMP-7 reverses EMT (Kalluri R and Zeisberg M (2003) Histol Histopathol 18:217-224.)

HGF inhibits EMT by antagonizing the TGF-β pathway by inducing the expression of the Smad-binding inhibitory protein SnoN (Yang J, et al. (2005) J Am Soc Nephrol 16:68-78.) Administration of hepatocyte growth factor blocks EMT and retards renal fibrosis in several animal models including models of diabetic nephropathy and obstructive nephropathy (Yang J, et al.(2003) Am J Pathol 163:621-632; Mizuno S and Nakamura T (2004) Am J Physiol Renal Physiol 286:F134-143.) However, when administered to an in vivo model, HGF only provided a partial reversal of EMT (Yang J and Liu Y (2003) Am J Physiol Renal Physiol 284:F349-357.) HGF has also been cited as an inducer of an EMT-like phenotype termed reversible scatter, therefore, it may not be the most straightforward agent for reversing EMT (Janda E, et al. (2002) J Cell Biol 156:299-313.)

Other reports of mesenchymal phenotype reversion utilized the EpH4 mouse mammary epithelial cells. EMT induced in EpH4 cells by a combination of activated Ras and TGF-β could be reversed by agents that block Ras function (Janda E, et al. (2002) J Cell Biol 156:299-313) or by a dominant negative protein inhibitor of NFkB signaling (Huber M A, et al. (2004) J Clin Invest 114:569-581.)

EMT also was induced in these cells by an estradiol-inducible c-Fos-estrogen receptor fusion protein that caused increased TGFβ1 expression and TGFβ signaling in the cells, as indicated by Smad nuclear localization and increased expression of TGFβ responsive reporter genes in response to estradiol. Reversal was accomplished by combining constitutive expression of E-cadherin together with a small molecule inhibitor of the TGF-β type I kinase (BIBU 3029) (Eger A, et al. (2004) Oncogene 23:2672-2680.) As in the present study, treatment of the mesenchymal cells with BIBU 3029 as a single agent for 3-6 days generated only a partial morphological reversion including the formation of cell-cell contacts and loss of nuclear beta-catenin, however the cells did not revert to a close packed epithelial morphology or express detectable E-cadherin. Ectopic E-cadherin caused the cells to form epithelial-like sheets of cells without fully assembled tight junctions, but this partial reversion did not occur in the presence of TGFβ1. Addition of BIBU 3029 to the E-cadherin expressing cells caused the formation of cobblestone-like epithelial sheets with tight junctions between the cells and localized expression of E-cadherin and beta-catenin at cell junctions. Junction formation occurred at about 50% of the cells after six days of treatment. It is unclear why the reversion took longer than the 24 hour treatment we used with TGF-β Type I receptor inhibitor and the Rho kinase inhibitor. This could simply be a difference between the two cell types, but it might indicate that more indirect processes are induced by ectopic E-cadherin. Establishing clonal lines of the mesenchymal cells expressing ectopic E-cadherin would have provided many cell generations for other changes to occur in the cells. In addition to inhibiting beta-catenin/LEF-TCF transcriptional activity, ectopic expression of E-cadherin also might aid in the reversion of EMT through a by-pass of the downregulation of the endogenous E-cadherin gene, a hallmark of EMT that is mediated by snail, slug, SIP1 or twist in different cell types. In the future, it will be important to test the cooperation of beta-catenin and TGF-β signaling for maintaining the mesenchymal state using selective cell-permeable chemical inhibitors of beta-catenin function.

In chronic fibrotic diseases, reversal of the mesenchymal state generated by EMT may be critical to restoring function to the organ and provide a potential treatment for chronic kidney damage caused by constitutive, high levels of TGF-β1 ligand. The role of TGF-β as a key mediator of fibrosis and facilitator of cancer growth and metastasis has stimulated the development of a number of therapeutic strategies, some of which are in clinical trials (Yingling J M, et al. (2004) Nat Rev Drug Discov 3:101.1-1022.) In addition, ROCK and p38 MAPK inhibitors are in clinical trials (Shimokawa H, et al. (2002) J Cardiovasc Pharmacol 40:751-761; Mohri M, et al. (2003) J Am Coll Cardiol 41:15-19.) The results presented here suggest that combinations of TGF-β inhibitors with ROCK inhibitors or p38 MAPK inhibitors may provide more effective therapeutic strategies than single kinase inhibitors in diseases in which EMT contributes to the pathology.

We claim:

1. A method of reversing epithelial mesenchymal transition, comprising the step of treating a fibrotic disease patient or cancer disease patient with an amount of kinase inhibitor capable of reversing EMT, wherein the kinase inhibitor comprises a TGF-βI kinase inhibitor and a Rho kinase inhibitor or a TGF-βI inhibitor and a p38 MAPK inhibitor.

2. The method of claim 1 wherein the administration of the inhibitors is simultaneous.

3. The method of claim 1 wherein the TGF-βI inhibitor is SB431542.

4. The method of claim 1 wherein the TGF-βI inhibitor is selected from the group consisting of SB431542, SD-208, SB-525334, SM16, and LY2157299.

5. The method of claim 1 wherein the Rho kinase inhibitor is Y27632.

6. The method of claim 1 wherein the Rho kinase inhibitor is Y27632 and the TGF-βI inhibitor is SB431542.

7. The method of claim 1 wherein the p38 MAPK inhibitor is selected from the group consisting of SB203580 and SB202190.

8. The method of claim 1 wherein the Rho kinase inhibitor is a statin.

9. The method of claim 1 wherein the disease is selected from the group consisting of diabetic nephropathy, liver cirrhosis, scleroderma, scarring, keloids and adhesions.

10. The method of claim 1 wherein the inhibitors are administered via the group consisting of tablets, coated tablets, capsules, pills, aqueous solutions, suspensions, emulsions, sterile injectable solutions, nonaqueous emulsions, suspensions and solutions, sprays and forms with protracted release of active compound.

11. A pharmaceutical composition comprising an amount of kinase inhibitor capable of reversing EMT, wherein the kinase inhibitor comprises a TGF-βI kinase inhibitor and a Rho kinase inhibitor or a TGF-βI inhibitor and a p38 MAPK inhibitor.

12. The composition of claim 11 wherein the composition is in the form of tablets, coated tablets, capsules, pills, aqueous solutions, suspensions, emulsions, sterile injectable solutions, nonaqueous emulsions, suspensions and solutions, sprays and forms with protracted release of active compound.

13. The composition of claim 11 wherein the TGF-βI inhibitor is selected from the group consisting of SB431542, SD-208, SB-525334, SM16, and LY2157299.

14. The composition of claim 11 wherein the Rho kinase inhibitor is Y27632.
